(19) United States
(12) Patent Application Publication
(10) Pub. No.: US 2020/0215284 A9
(43) Pub. Date: Jul. 9, 2020

(54) AIRWAY DEVICE

(71) Applicant: E-PROSPERITY & DEVELOPMENT CO., Ltd., Taipei City (TW)

(72) Inventors: Cheng-Shih Lien, Taipei City (TW); Tsu-Tai Lin, Taipei City (TW); Ming-Thau Sheu, Taipei City (TW); Chen-Chun Liao, Taipei City (TW)

(73) Assignee: E-PROSPERITY & DEVELOPMENT CO., Ltd., Taipei City (TW)

(21) Appl. No.: 15/851,743

(22) Filed: Dec. 22, 2017

Related U.S. Application Data
(60) Provisional application No. 62/437,691, filed on Dec. 22, 2016.

(51) Int. Cl. A61M 16/04; A61L 29/14; A61L 29/06

(52) U.S. Cl. CPC A61M 16/0438; A61M 16/044; A61M 2210/1028; A61L 29/145; A61L 29/06; A61M 16/0447

(57) ABSTRACT

An airway device for a subject which has an airway tube having a distal end and a proximal end, and a laryngeal mask disposed surrounding the distal end of the airway tube, wherein the inner part of the laryngeal mask has a hollow body filled with a biocompatible thermosensitive hydrogel, wherein the biocompatible thermosensitive hydrogel is gelified within a temperature range such that the biocompatible thermosensitive hydrogel, upon being gelified, allows the laryngeal mask to fit over the circumference of a laryngeal inlet of the subject.

The present application claims priority to U.S. Provisional Application No. 62/473,691, filed on Dec. 22, 2016, which is incorporated herein by reference in its entirety.

### BACKGROUND OF THE INVENTION

### Field of the Invention

The present invention relates to an airway device and is particularly applicable, but in no way limited, to a laryngeal airway devices having a laryngeal mask and the inner hollow body of the laryngeal mask is filled with a biocompatible thermosensitive hydrogel.

### Description of Prior Art

A wide variety of devices generally known as the laryngeal mask airway (LMA) due to its ease of operability are currently used in patients spontaneously breathing under anaesthesia, in patients recovering after anaesthesia, or in weaning patients in intensive care, during resuscitation of patients to provide a clear and hands-free airway.

Basically there are two types of LMA, one with an inflatable sealing cuff and the other with a non-inflatable sealing cuff. In FIG. 1, first type of LMA 10P with an inflatable sealing cuff comprises a flexible tubular portion 11P, an inflatable sealing cuff 13P, and an inflation tube 15P. The flexible tubular portion 11P comprises a first opening part 111P and a second opening part 112P. The inflatable sealing cuff 13P is disposed surrounding the first opening part 111P. The inflation tube 15P has one end connected to the inflatable sealing cuff 13P and the other end directing gas into the inflatable sealing cuff 13P. FIGS. 2 to 4 are illustrative diagrams showing the conventional laryngeal mask in use. During the installation of the LMA 10P, operators always have to insert their finger(s) into a subject's mouth to pass the soft, inconvenient LMA 10P through the upper jaw because of the obstruction caused by the subject's tongue. On the other hand, the inflated sealing cuff 13P after being aspirated with gas might not fit anatomically with and form a seal over the laryngeal structure of the subject potentially causing gas leakage.

Second type of LMA airway device comprises an airway tube having a distal end and a proximal end, and the distal end of which is surrounded by a laryngeal cuff, wherein the cuff is non-inflatable and pre-formed in a shape as described in GB2393993A and U.S. Pat. No. 7,806,119. The laryngeal cuffs of these devices are generally non-inflatable, and formed from a soft, deformable material adaptable to the individual detail of the subject's laryngeal inlet to form a satisfactory gas-tight seal. Because of the very soft, deformable nature of the laryngeal cuff, it is essential to maintain the laryngeal cuff in a gas-tight contact with the laryngeal inlet at all times while in use. However, since such device also comprises several solid special materials which increase both weight and costs, and cause disadvantages for practical applications of LMA, particularly where the item is intended for single use or as a disposable item.

Therefore, it is desirable to provide a cost effective laryngeal mask airway device for mitigating and/or obviating the aforementioned problems associated with these two types of LMA.

### SUMMARY OF THE INVENTION

The present invention provides an airway device for a subject comprising an airway tube having a distal end and a proximal end, and a laryngeal mask disposed surrounding the distal end of the airway tube, wherein the inner part of the laryngeal mask has a hollow body filled with a biocompatible thermosensitive hydrogel, wherein the biocompatible thermosensitive hydrogel is gelified within a temperature range such that the biocompatible thermosensitive hydrogel, upon being gelified, allows the laryngeal mask to fit over the circumference of a laryngeal inlet of the subject.

### BRIEF DESCRIPTION OF THE DRAWINGS

FIGS. 1 to 4 are illustrations of a laryngeal mask airway (LMA) used in the prior art.

FIG. 5 shows the structure of the airway device of the present invention.

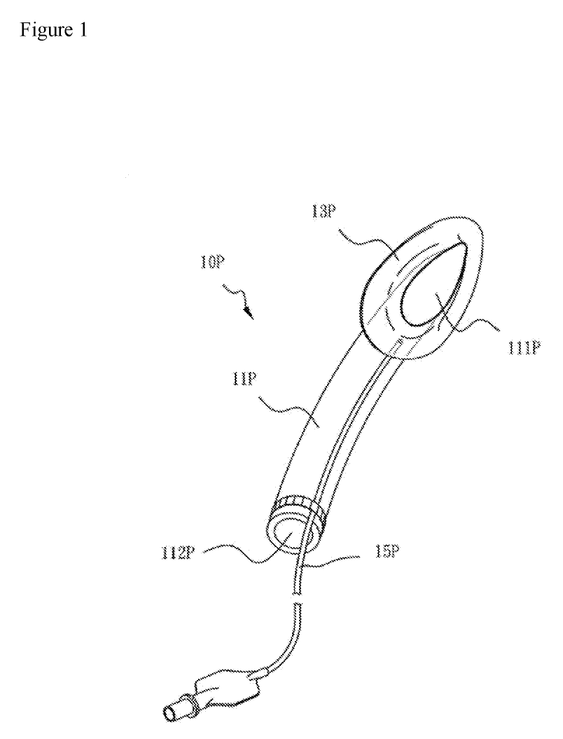

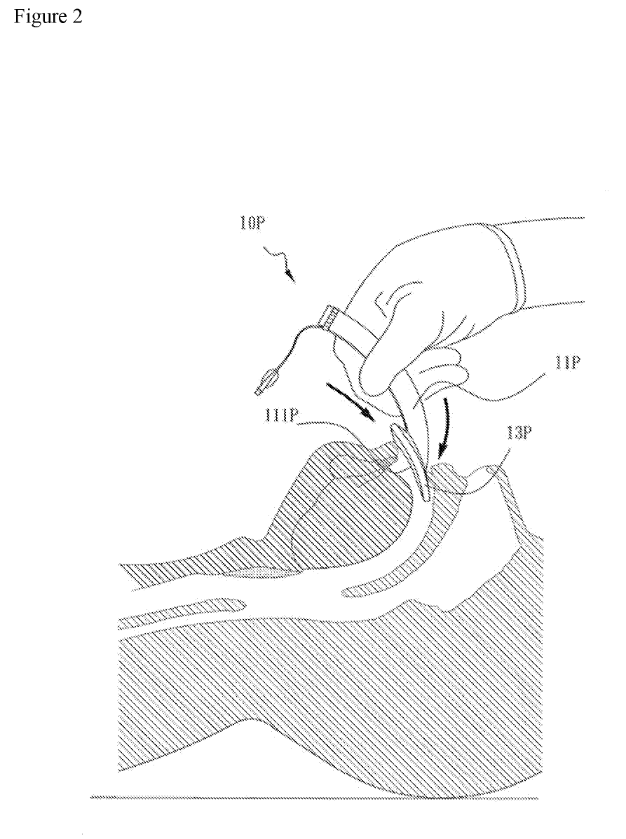

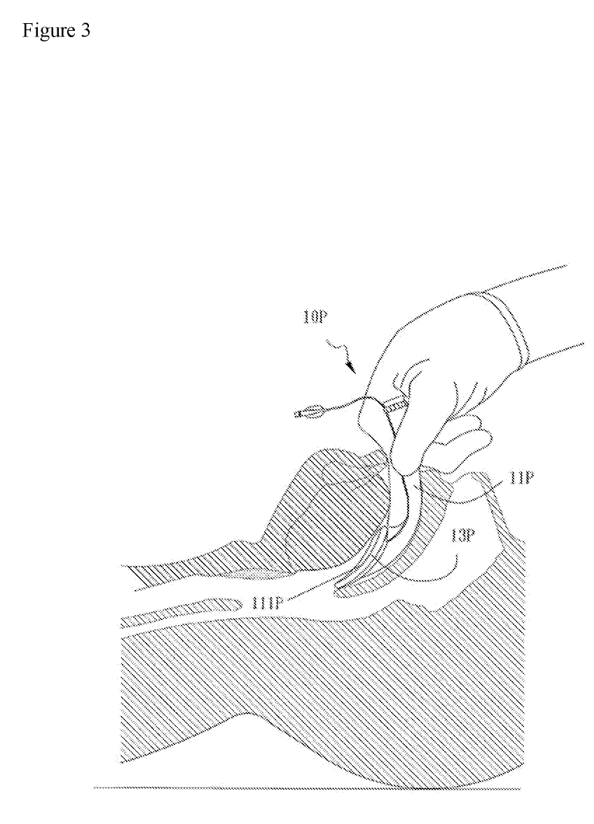

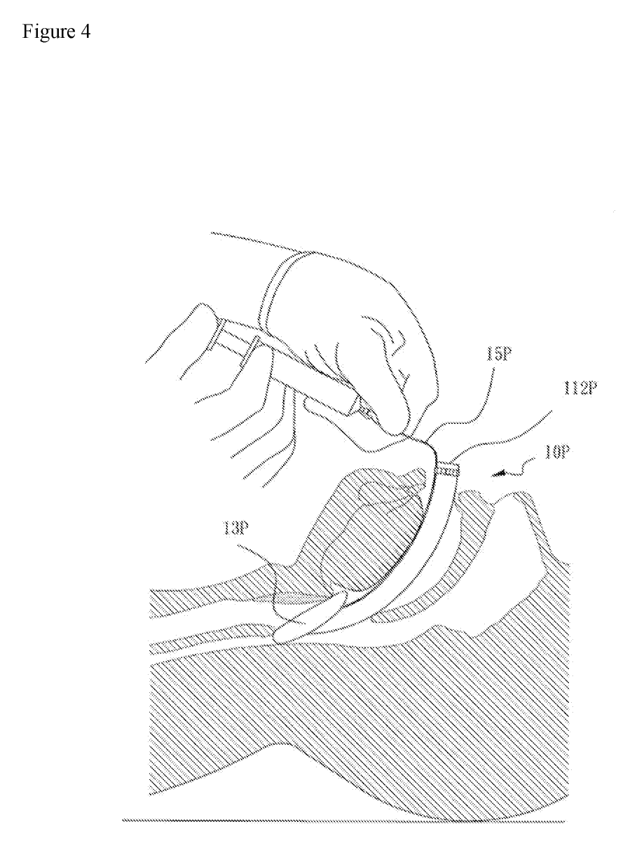

### DETAILED DESCRIPTION OF THE INVENTION

This invention provides a laryngeal mask of an airway device filled with a thermosensitive hydrogel. The laryngeal mask is able to achieve a gas-tight seal between the mask and a laryngeal inlet of a subject. Before contacting the laryngeal inlet, the thermosensitive hydrogel is in a solution form, which is soft and deformable and filled in the inner hollow body of the laryngeal mask, allows the laryngeal mask to be located in a proper position with easiness during insertion. As soon as the thermosensitive hydrogel in the laryngeal mask contacts the subject's laryngeal inlet, the filled thermosensitive hydrogel is transformed from solution form to gel form when sensing the subject's body temperature, which enables the laryngeal mask of the present invention to firmly conform to the structure of the laryngeal with a gas-tighter seal than the cuff inflated with air is able to. With the filling of thermosensitive hydrogel in the laryngeal mask, the drawbacks of conventional LMA with gas-filled cuff can be eliminated and the material cost of the non-inflatable and pre-formed type LMA can be reduced. The airway device in this invention is thus a lower cost and more effective LMA intended for a single use or as a disposable item that can be manufactured.

As used throughout, the singular forms “a,” “an,” and “the” include plural referents unless the context clearly dictates otherwise.

The term “or” as used herein may mean “and/or.”

The present invention provides an airway device for a subject comprising an airway tube having a distal end and a proximal end, and a laryngeal mask disposed surrounding the distal end of the airway tube, wherein the inner part of the laryngeal mask has a hollow body filled with a biocompatible thermosensitive hydrogel, wherein the biocompatible thermosensitive hydrogel is gelified within a temperature range such that the biocompatible thermosensitive hydrogel, upon being gelified, allows the laryngeal mask to fit over the circumference of a laryngeal inlet of the subject.

As used herein, the term “subject” refers to a mammal, preferably a human. The mammals include, but are not limited to, humans, primates, livestock, rodents, and pets.

The airway device may be constructed from any suitable plastics material as selected by material specialists. Latex-free medical grade silicone rubber is one preferred material. The laryngeal mask should be soft in texture to avoid undue damages to the surrounding tissues. Other suitable materials for construction of this type of the airway device include, but are not limited to, Poly Vinyl Chloride (PVC), Thermoplastic Elastomers such as the styrenic block copolymers (e.g., Styrene Butadiene Styrene (SBS) and Styrene Ethylene Butylene Styrene (SEBS)), Thermoplastic Olefin Blends (TPO), Thermoplastic PolyUrethanes (TPU), Thermoplastic Vulcanisates (TPV), Copolyester (COPE), Polyether Block Amides (PEBAX), Melt Processable Rubbers, Flexible Co-polymers (e.g., EVA), and foamed versions thereof, where appropriate. In one embodiment, the airway tube and the laryngeal mask are formed of a flexible polymer material for medical use.

A further important factor involved in the choice of a suitable material is transparency. Ideally, the materials of construction should be substantially clear or transparent. This enables anaesthetists or operators to see the inner lumen of the airway to check for blockages or other problems. Such transparent materials are well known to material specialists.

In one embodiment, the profile of the airway tube is substantially circular. In an alternative embodiment the profile of the airway tube is substantially elliptical. In another embodiment, the airway tube is used for supplying gas, such as oxygen.

In another embodiment, the airway tube is formed by an extrusion process.

In one embodiment, the distal end of the airway tube has a first opening part. In another embodiment, the proximal end of the airway tube has a second opening part. Therefore, the laryngeal mask is disposed surrounding the first opening part.

In another embodiment, the laryngeal mask is a laryngeal cuff or a mask body. The hollow body is encapsulated inside the laryngeal mask. The laryngeal mask comprises a ventral region and a dorsal region. The ventral region of the laryngeal mask is sized and shaped to fit over the circumference of the laryngeal inlet of the subject when using. In a preferred embodiment, the ventral region of the laryngeal mask is substantially concave in shape.

In one embodiment, the ventral region of the laryngeal mask is adapted to form an anatomical fit over the laryngeal inlet of the subject and is provided with protuberances which form a tight seal with the pyriform fossae and aryepiglottic folds of the laryngeal inlet of the subject. In a preferred embodiment, the ventral region of the laryngeal mask is adapted to form an anatomical fit over the laryngeal inlet of the subject and is provided with protuberances which form a tight seal with the vallecula, epiglottis, aryepiglottic folds, pyriform fossae, and around the thyroid and cricoid cartilages. In a preferred embodiment, the ventral region of the laryngeal mask is adapted to fit anatomically over the laryngeal framework of the subject is provided with grooves to allow passage of vital arteries, veins and nerves supplying the laryngeal framework.

In an alternative embodiment the laryngeal mask and the airway tube are formed from material of substantially the same shore hardness.

With respect to the softness (hardness) of the laryngeal mask, on the Shore A scale of Hardness, a hardness of less than 30 for the ventral region of the laryngeal mask that contacts the laryngeal inlet is optimum. Further, a value on the same scale of between 0 to 20 is preferred, with a particularly preferred range of 0 to 5. The apparent softness of the laryngeal mask can be further adapted by forming cavities or channels within the body of the mask itself.

The term “hydrogel” as used herein refers to a three-dimensional, hydrophilic or amphiphilic polymeric network capable of taking up large quantities of water. The networks are composed of homopolymers or copolymers, and are insoluble due to the presence of covalent chemical or physical (ionic, hydrophobic interactions, entanglements) crosslinks. The crosslinks provide the network structure and physical integrity. Hydrogels exhibit a thermodynamic compatibility with water that allows them to swell in aqueous media.

The term “crosslink” as used herein refers to a composition containing intramolecular and/or intermolecular crosslinks, whether arising through covalent or noncovalent bonding. “Noncovalent” bonding includes both hydrogen bonding and electrostatic (ionic) bonding.

The term “thermosensitive hydrogel” as used herein refers to a polymer of the present disclosure, which forms, to various degrees, a jelly-like or gelled product when heated to a particular temperature, for example body temperature (37° C.), or a temperature higher than 25° C. The polymer is preferably a liquid at room temperature and soluble in water, but upon reaching a particular temperature, forms a hydrogel when mixed with water such that water is a dispersion medium forming the hydrogel.

In one embodiment, the biocompatible thermosensitive hydrogel is gelified to form a gel. In another embodiment, the biocompatible thermosensitive hydrogel has a gel formation temperature from 10° C. to 40° C. In a preferred embodiment, the biocompatible thermosensitive hydrogel has a gel formation temperature from 20° C. to 35° C. In a more preferred embodiment, the biocompatible thermosensitive hydrogel has a gel formation temperature from 25° C. to 35° C.

In another embodiment, the biocompatible thermosensitive hydrogel is composed of a polymer. In an embodiment, the biocompatible thermosensitive hydrogel is prepared by dissolving a biocompatible thermosensitive polymer in water, buffer, or a mixture of water and cosolvent. In a preferred embodiment, the cosolvent is selected from alcohol, glycerin, polyethylene glycol, propylene glycol, or any combination thereof.

The term “polymer” as used herein is defined as a compound comprising a linear arrangement of simpler repeating molecules.

In one embodiment, the polymer is selected from poly (ethylene oxide-propylene oxide-ethylene oxide) (PEO-PPO-PEO); methyl cellulose; N-isopropylacrylamide (NiPAAM); poly(vinyl alcohol); poly(NiPAAM)/poly(ethylene glycol); poly(ethylene glycol-lactic acid-ethylene glycol) (PEG-PLLA-PEG), or any combination thereof. In a preferred embodiment, the poly (ethylene oxide-propylene oxide-ethylene oxide) (PEO-PPO-PEO) comprises pluronic acid. In a more preferred embodiment, the pluronic acid comprises Pluronic F68, Pluronic F127, Pluronic F108, Pluronic F38, and Pluronic F87. In a more preferred embodiment, the pluronic acid is Pluronic F127.

In another embodiment, the biocompatible thermosensitive hydrogel is prepared by dissolving the pluronic acid in the water. In a preferred embodiment, the concentration of the pluronic acid is 10-30 wt %. In a preferred embodiment, the concentration of the pluronic acid is 20-30 wt %.

In one embodiment, the polymer comprises the pluronic acid and poly ethylene glycol (PEG). In a preferred embodiment, the PEG is polyethylene glycol 6000. In another embodiment, the biocompatible thermosensitive hydrogel is prepared by adding the pluronic acid and the PEG in the water. In a preferred embodiment, the concentration of the PEG is 1-5 wt %. In a more preferred embodiment, the concentration of the PEG is 1-3 wt %.

In one embodiment, the airway device further comprises an infusion tube, which is connected to the hollow body of the laryngeal mask for filling the biocompatible thermosensitive hydrogel into the hollow body. In another embodiment, the laryngeal mask further has a pore for allowing injection of the biocompatible thermosensitive hydrogel into the hollow body. In a preferred embodiment, the infusion tube has one end connected to the pore of the laryngeal mask and the other end directing the biocompatible thermosensitive hydrogel into the hollow body.

The present invention also provides a kit of airway device comprising an airway device comprising an airway tube having a distal end and a proximal end, and a laryngeal mask disposed surrounding the distal end of the airway tube, wherein the inner part of the laryngeal mask has a hollow body used for filling with a biocompatible thermosensitive hydrogel; and a container containing the biocompatible thermosensitive hydrogel.

In one embodiment, the distal end of the airway tube has a first opening part. In another embodiment, the proximal end of the airway tube has a second opening part. Therefore, the laryngeal mask is disposed surrounding the first opening part. In another embodiment, the airway tube is used for supplying gas, such as oxygen.

In another embodiment, the laryngeal mask comprises a ventral region and a dorsal region. The ventral region of the laryngeal mask is sized and shaped to fit over the circumference of the laryngeal inlet of the subject when using. In a preferred embodiment, the ventral region of the laryngeal mask is substantially concave in shape.

In one embodiment, the airway device further comprises an infusion tube, which is connected to the hollow body of the laryngeal mask for filling the biocompatible thermosensitive hydrogel into the hollow body.

When used clinically, the biocompatible thermosensitive hydrogel can be filled into the hollow body of the laryngeal mask before the laryngeal mask is inserted into a oral cavity of a subject; or the biocompatible thermosensitive hydrogel is filled into the hollow body of the laryngeal mask when the laryngeal mask gets to a specific position around the laryngeal opening of the subject. Based on the property of the biocompatible thermosensitive hydrogel which is gelified within a temperature range, the biocompatible thermosensitive hydrogel is gelified by contacting with the body temperature of the subject so as to make the laryngeal mask fit over the circumference of the laryngeal inlet of the subject.

In one embodiment, the temperature range is 10-40° C. In a preferred embodiment, the temperature range is 20-35° C. In a more preferred embodiment, the temperature range is 25-35° C.

Thus, the airway device of the present invention is both efficient in clinical operation and cost-effective to be manufactured.

### EXAMPLES

The present invention may be embodied in different forms, and is not limited to the examples mentioned below. The following examples are merely representative of various aspects and features of the present invention.

### Example 1: Preparation of Thermosensitive Hydrogel

A. Method:

(1) Preparation of Thermosensitive Hydrogel with 20% Pluronic F127

The thermosensitive hydrogel of Pluronic F127 (PF127) was prepared by a physical mixing method as follows: 80 gram of deionized water and 20 grams of PF127 were mixed together and put in a refrigerator overnight until a clear solution was formed. The sol-gel transition temperature of the resulting hydrogel was measured to be in the range of 10-30° C. Airway leak pressure>25 cm H₂O.

(2) Preparation of Thermosensitive Hydrogel with 25% Pluronic F127

The thermosensitive hydrogel of PF127 was prepared by a physical mixing method as follows: 75 gram of deionized water and 25 grams of PF127 were mixed together and put in a refrigerator overnight until a clear solution was formed. The sol-gel transition temperature of the resulting hydrogel was measured to be in the range of 10-30° C. Airway leak pressure>25 cm H₂O.

(3) Preparation of Thermosensitive Hydrogel with 30% Pluronic F127

The thermosensitive hydrogel of PF127 was prepared by a physical mixing method as follows: 70 gram of deionized water and 30 grams of PF127 were mixed together and put in a refrigerator overnight until a clear solution was formed. The sol-gel transition temperature of the resulting hydrogel was measured to be in the range of 5-25° C. Airway leak pressure>25 cm H₂O.

(4) Preparation of Thermosensitive Hydrogel with 20% Pluronic F127 and with Addition of 1% PEG 6000

The thermosensitive hydrogel of PF127 was prepared by a physical mixing method as follows: 80 gram of deionized water, 20 grams of PF127, 1 gram of PEG 6000 were mixed together and put in a refrigerator overnight until a clear solution was formed. The sol-gel transition temperature of the resulting hydrogel was measured to be in the range of 20-40° C. Airway leak pressure>25 cm H₂O.

(5) Preparation of Thermosensitive Hydrogel with 25% Pluronic F127 and with Addition of 2% PEG 6000

The thermosensitive hydrogel of PF127 was prepared by a physical mixing method as follows: 75 gram of deionized water, 25 grams of PF127, and 2 gram of PEG 6000 were mixed together and put in a refrigerator overnight until a clear solution was formed. The sol-gel transition temperature of the resulting hydrogel was measured to be in the range of 25-40° C. Airway leak pressure>25 cm H₂O.

(6) Preparation of Thermosensitive Hydrogel with 30% Pluronic F127 and with Addition of 3% PEG 6000

The thermosensitive hydrogel of Pluronic F127 was prepared by a physical mixing method as follows: 70 grams of deionized water, 30 grams of PF127, and 3 grams of PEG 6000 were mixed together and put in a refrigerator overnight until a clear solution was formed. The sol-gel transition temperature of the resulting hydrogel was measured to be in the range of 25-40° C. Airway leak pressure>25 cm H₂O.

B. Assay:

Rheological Characterization of Hydrogel

The rheological parameters were measured by a rheometer (HAAKE Rotation Rheometer RS-1, Germany). The test method employed was dynamic temperature ramp. The rheological parameters changing with temperature were measured at a fixed frequency in the range of 1-20 Hz, in the temperature range of 10-60° C., and at a heating rate of 1.5° C./min (Q. Wang, H. B. Xu, X. L. Yang, Y. J. Yang, Rheological Study of Aqueous Dispersions of In Situ Gelable Thermosensitive Polymer Nanogels, Polym Eng Sci, 49 (2009) 177-181).

### Example 2: Use of the Airway Device

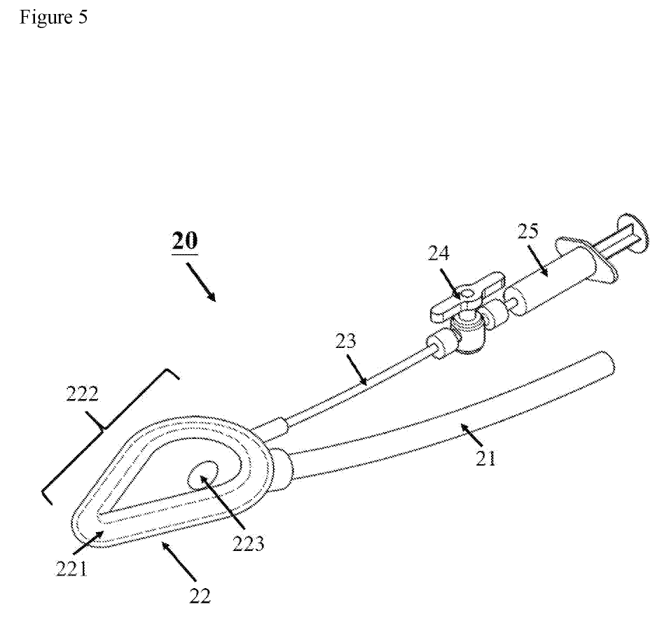

As shown in FIG. 5, the airway device 20 of the present invention comprised an airway tube 21 having a distal end and a proximal end, and a laryngeal mask 22 disposed surrounding the distal end of the airway tube 21, wherein the inner part of the laryngeal mask 22 had a hollow body 221 filled with a biocompatible thermosensitive hydrogel, wherein the biocompatible thermosensitive hydrogel was gelified within a temperature range such that the biocompatible thermosensitive hydrogel, upon being gelified, allowed the laryngeal mask 22 to fit over a circumference of a laryngeal inlet of a subject.

In addition, the airway device 20 further comprised an infusion tube 23 which is connected to the hollow body 221 of the laryngeal mask 22 for filling the biocompatible thermosensitive hydrogel into the hollow body 221. The distal end of the infusion tube 23 might have a valve 24 for controlling the input of the biocompatible thermosensitive hydrogel. Therefore, the biocompatible thermosensitive hydrogel was first filled into a syringe 25, and then the syringe 25 was connected to the valve 24 and the infusion tube 23 when using. In an appropriate condition, the solution form of the biocompatible thermosensitive hydrogel in the syringe 25 was inputted into the hollow body 221 of the laryngeal mask 22 via the infusion tube 23.

The laryngeal mask 22 with the hollow body 221 and the airway tube 21 of the airway device 20 were composed of a flexible silicone-rubber polymer, and the laryngeal mask 22 had an elliptical cross-section, approximating, for ease of insertion, the shape of a human throat. Being capable of spreading radially, the airway tube 21 could accommodate the passage of cuffed endotracheal tubes and related medical devices up to 8.0 French in diameter. The length of the airway tube 21 was such that when the laryngeal mask 22 was properly positioned for use within the oropharynx, the proximal end of the airway tube 21 was located exterior to the oral cavity. In alternative embodiments, the proximal end of the airway tube 21 might be fitted with adapters or connectors suitable for connection to a variety of medical devices, for example, lung ventilation machines, secured to the proximal end of the airway tube 21, forming a smooth arcuate curve.

The airway tube 21, which was in the form of a hollow cylinder of substantially uniform cross-section open at each end, extended from the medical device end into the laryngeal mask 22 to connect with a concave opening 223 in a ventral region 222 of the laryngeal mask 22. The inside diameter of the airway tube 21, depending on the size of the airway device 20, is generally larger in adult sizes versus pediatric sizes and designed in general to accommodate the appropriately sized endotracheal tube for endoscope guided intubation where necessary. The internal diameter of the airway tube 21 might be substantially uniform along its length, although the internal diameter of the airway tube 21 might vary.

The ventral region 222 of the laryngeal mask 22 had a concave opening 223 and the dorsal region of the laryngeal mask 22 was closed. The concave opening 223 in the ventral region 222 of the laryngeal mask 22 was connected directly to the airway tube 21 such that air/gas was free to flow from the medical devices through the airway tube 21 and out of the concave opening 223 in the ventral region 222 of the laryngeal mask 22.

In a further embodiment, the laryngeal mask 22 was formed around a pre-formed or pre-cut piece of the airway tube 21. In addition, the airway tube 21 may be circular in profile or elliptical or any other profile as selected by designers.

The laryngeal mask 22 had a pore (not shown in FIG. 5) for allowing injection of the biocompatible thermosensitive hydrogel into the hollow body 221. The infusion tube 23 was connected to the pore of the laryngeal mask 22. Therefore, a user could inject the biocompatible thermosensitive hydrogel into the hollow body 221 of the laryngeal mask 22 via the infusion tube 23. Further, the user could select one of the following three conditions to fill the hollow body 221 with biocompatible thermosensitive hydrogel: (1) filling the hollow body 221 of the laryngeal mask 22 with the solution form of the biocompatible thermosensitive hydrogel before inserting the laryngeal mask 22 into the oral cavity of the subject; (2) injecting the solution form of the biocompatible thermosensitive hydrogel into the hollow body 221 of the laryngeal mask 22 via the pore when the laryngeal mask 22 gets to a specific position around the laryngeal opening of the subject; or (3) filling the hollow body 221 of the laryngeal mask 22 with a biocompatible thermosensitive polymer before inserting the laryngeal mask 22 into the oral cavity of the subject, and then injecting water into the hollow body 221 via the pore to make the biocompatible thermosensitive polymer form the biocompatible thermosensitive hydrogel when the laryngeal mask 22 got to a specific position around the laryngeal opening of the subject. After the laryngeal mask 22 was in the proper positioning of the anatomy surrounding the laryngeal opening, the biocompatible thermosensitive hydrogel in the hollow body 221 of the laryngeal mask 22 would sense the subject's body temperature and transform from solution form to gel form, allowing the laryngeal mask 22 of the airway device 20 of the present invention to form a tight seal covering the laryngeal opening.

### Example 3 Comparison of Airway Devices

The use of laryngeal mask airway (LMA) devices was popular. Most LMAs were air-filled cuff type. Therefore, the present invention compared the function of the air-filled LMA with that of the LMA filled with thermosensitive hydrogel of the present invention.

The present invention used the thermosensitive hydrogel prepared with 20% Pluronic F127 to perform the oropharyngeal leak pressure (OLP) test. OLP of the airway device was determined by closing the expiratory valve of the circle system at a fix gas inflow of 3 L/min. A higher OLP was a marker of efficacy and safety when using airway devices. As shown in table 1, the LMA filled with thermosensitive hydrogel of the present invention had higher OLP compared to the air-filled LMA. Complications related to airway devices and thermo-sensitive hydrogel were not seen during the test.

TABLE 1
Comparison of airway devices

| | OLP |
|---|---|
| air-filled LMA | >20 cm H₂O |
| LMA filled with | >25 cm H₂O |
| thermosensitive hydrogel | |

The above description is the specific description of one preferred embodiment of the present invention, which is not intended to be limiting. Accordingly, any variations and modifications of the invention made by one skilled in the art within the scope and spirit of the invention are all within the scope of the invention.

What is claimed is:

1. An airway device for a subject comprising an airway tube having a distal end and a proximal end, and a laryngeal mask disposed surrounding the distal end of the airway tube, wherein the inner part of the laryngeal mask has a hollow body filled with a biocompatible thermosensitive hydrogel, wherein the biocompatible thermosensitive hydrogel is gelified within a temperature range such that the biocompatible thermosensitive hydrogel, upon being gelified, allows the laryngeal mask to fit over the circumference of a laryngeal inlet of the subject.

2. The airway device of claim 1, wherein the ventral region of the laryngeal mask is adapted to form an anatomical fit over the laryngeal inlet of the subject and is provided with protuberances which form a tight seal with the pyriform fossae and aryepiglottic folds of the laryngeal inlet of the subject.

3. The airway device of claim 1, wherein the laryngeal mask is composed of a soft deformable material.

4. The airway device of claim 1, wherein the biocompatible thermosensitive hydrogel is composed of a polymer which comprises pluronic acid.

5. The airway device of claim 1, wherein the biocompatible thermosensitive hydrogel is composed of a polymer which comprises pluronic acid and poly ethylene glycol (PEG).

6. The airway device of claim 1, wherein the temperature range is 10-40° C.

7. The airway device of claim 1, wherein the temperature range is 20-35° C.

8. The airway device of claim 1, wherein the temperature range is 25-35° C.

9. The airway device of claim 1, wherein the subject is a human.

10. The airway device of claim 1 further comprising an infusion tube which is connected to the hollow body of the laryngeal mask for filling the biocompatible thermosensitive hydrogel into the hollow body.
